(19) United States
(12) Patent Application Publication
(10) Pub. No.: US 2008/0199959 A1
(43) Pub. Date: Aug. 21, 2008

(54) Method For Cell Culture

(75) Inventors: Mattias Algotsson, Uppsala (SE); Gunnar Glad, Uppsala (SE); Nicolas Thevenin, Uppsala (SE)

Correspondence Address: GE HEALTHCARE BIO-SCIENCES CORP.;PATENT DEPARTMENT, 800 CENTENNIAL AVENUE, PISCATAWAY, NJ, 08855, US

(73) Assignee: GE HEALTHCARE BIO-SCIENCES AB, UPPSALA (SE)

(21) Appl. No.: 11/917,169

(22) PCT Filed: Jun. 19, 2006
(86) PCT No.: PCT/SE2006/000750

§ 371 Date: Dec. 11, 2007

(30) Foreign Application Priority Data
Jun. 21, 2005 (SE) 0501513-6
Jun. 19, 2006 (SE) PCT/SE2006/000750

(51) Int. Cl. C12N 5/06; C12N 1/20; C12N 1/00; C12M 1/00

(52) U.S. Cl. 435/402; 435/395; 435/252.1; 435/255.1; 435/287.1

(57) ABSTRACT

The present invention relates to a method for cell culture, more precisely small scale cell culture. In the present invention a screening tool is used which comprises particulate matter or microcarriers, such as beads, attached to a solid support, such as a microtiter plate, for the cultivation of cells on said microcarriers. The microcarriers are preferably cultivation beads, such as CYTODEX™. According to the invention, this small scale format for cell cultivation may be used for any testing involving cells, for example testing of optimal growth conditions for a specific type of cell, such as stem cells. Another use is cell expansion.

### CROSS-REFERENCE TO RELATED APPLICATIONS

This application is a filing under 35 U.S.C. § 371 and claims priority to international patent application number PCT/SE2006/000750 filed Jun. 19, 2006, published on Dec. 28, 2006, as WO 2006/137787, which claims priority to Swedish patent application number 0501513-6 filed Jun. 21, 2005.

### FIELD OF THE INVENTION

The present invention relates to a method for cell culture, more precisely small scale cell culture. In the present invention use is made of particulate matter, such as beads or fibers, attached to a solid support, such as a microtiter plate, for the cultivation of cells on said particles. According to the invention, a screening tool is provided for small scale cell cultivation. The screening tool may be used for any testing involving cells, for example testing of optimal growth conditions for a specific type of cell, such as stem cells.

### BACKGROUND OF THE INVENTION

Cell culture techniques have become vital to the study of animal cell structure, function and differentiation and for the production of many important biological materials, such as vaccines, enzymes, hormones, antibodies, interferons and nucleic acids. Another important area for cell culture is cell expansion from a small to a large cell population.

For cell culture it is conventional to grow the cells on a cell adhering surface since most mammalian cells and certain other cells are anchorage-dependent to be able to grow. Conventional cell culture in tissue culture treated bottles or other vials give a limited yield of anchorage-dependent cells due to the small surface areas available.

Microcarrier culture introduces new possibilities and for the first time makes possible the practical high yield culture of anchorage-dependent cells. In microcarrier culture cells grow as monolayers on the surface of small spheres which are usually suspended in culture medium by gentle stirring. By using microcarriers in simple suspension culture systems it is possible to achieve yields of several million cells per millilitre and the systems are easily scalable.

In the microcarrier approach, cell culture is realised with beads in a spinner flask or beads packed in columns (perfusion culture). The microcarriers are for example dextran, cellulose or polyethylene based products.

JP 09023876A describes a supporting material for cell culture capable of peeling cultured cells. A temperature sensitive polymer, for example poly N-substituted acrylamide, is coated on a plastic support and the cells are peeled off from the support by a temperature change affecting the coating. The coating is a thin grafted polymerized film on the plastic support.

WO 1994/011421 describes a method of modifying a polymer based surface with particles comprising converting the top layer of the polymer based surface to a swollen or semi swollen state without the use of adhesive and simultaneously or subsequently contacting the polymer based surface with the particles. Cell culture is not mentioned.

In the cell cultivation field, a faster screening of ligands, base matrices and cell culture conditions could be realised incredibly faster if the experiments could be scaled down. Thus more prototypes could be tested in the same period of time. This is not taught by the cell cultivation methods and devices according to prior art.

### SUMMARY OF THE INVENTION

The present inventors have found an entirely new way of culturing cells, namely to culture cells on microcarriers attached to a solid phase. According to the invention, the microcarriers are immobilised to the solid phase or support surface and in this way the outcome of the cell culture can easily be read directly from the solid phase or readout surface, for example in a microscope or other type of reading instrument. If the purpose is to expand cells, such as stem cells, the cells can be released from the support, for example if they are intended for therapy. Furthermore, work such as different assays may also be directly performed on cells attached to the surface.

Currently the testing of microcarriers, cell conditions and ligands as well as other factors for cell growth is usually performed in a 50 mL scale. Due to the scale of the synthesis and testing, only a few prototypes could be realised per day. It would be desirable to increase this number. The new way of culturing cells according to the invention enables small scale culturing and thereby small scale testing of different cell culture conditions.

According to the invention cell culture conditions on existing and new media can be optimized on a smaller scale and in parallel before a large scale process.

The present invention provides a method for cell culture comprising adding cells (in appropriate cell culture media) to microcarriers or particulate matter, such as beads or fibers, attached to a solid phase; and growing said cells on said microcarriers or particulate matter for small scale culture of cells. A purpose of this screening tool for cell culture is to test cell culture conditions, such as different microcarriers, ligands, culture media etc. Another purpose is to test cell culture material. A third purpose is cell expansion.

A preferred shape of the particles is as substantially spherical beads to give a large surface area for the cells to adhere onto. Another preferred shape is as elongate fibers. The particles may be beads or fibers made of synthetic or natural polymers or inorganic materials. Examples are dextran based beads, such as CYTODEX™, agarose based beads, such as SEPHAROSE™, polystyrene beads, such as SOURCE™, cross linked cellulose beads, such as CYTOPORE™, titania beads or silica beads. Different culture beads could be tested for the ones which are most suitable for the culture of a specific cell.

Optionally, the beads are coated with an adhesion factor, such as gelatine, fibronectin, laminin, collagen, vitronectin or tenascin,

The solid phase may be a molded article of any shape, such as a container, a Petri dish, a multiwell plate, a microtiter plate, a stick, a comb, a test tube, an Eppendorf tube, a sheet, a film etc. The molded article is preferably made of a synthetic polymer.

In one embodiment, the cell screening tool is a microtiter plate provided with immobilised culture beads in the wells of the microtiter plate.

The solid phase or support surface is made of or coated with polystyrene, styrene-acrylonitrile copolymer, styrene maleic anhydride copolymer, poly vinyl chloride resin etc.

The particulate matter may be attached to the solid phase by mechanical interlocking and/or interdiffusion of polymer chains. In this case, preferably the solid phase comprises a surface or coating capable of being at least partially dissolved/swollen in a solvent giving a viscous and tacky character that promotes adhesion of any added particulate matter.

Alternatively, the particles/spheres are attached to the solid phase by chemical bonding to the solid support. Biological bonding is also possible.

In a further alternative, the particles/spheres are attached to the solid phase by (hydrophobic) interaction to the solid support.

In yet a further alternative, the particles/spheres are bonded by a liquid adhesive, for example epoxy resin, to an inert surface, such as glass.

The present invention concerns any cell culture, i.e. culture of mammalian, bacterial or yeast cells, but preferably the cells are mammalian cells.

The mammalian cells may for example be embryonic or adult stem cells.

Optionally, the beads are provided with ligands having affinity for specific cells or cell structures, such as different cell receptors. The ligands may be synthetic or natural.

In a preferred embodiment, the method involves use is of a microtiter plate with a plurality of wells, such as 4, 16, 32 or 96, each provided with attached particles/spheres. Preferably dextran or agarose beads are used.

For study of cell culture conditions, cell growth or other parameters, the wells in the screening tool may have a different content. The particles/spheres may differ from each other in one or more of the wells of the microtiter plate or they may be the same. The culture conditions may also be different in one or more of the wells.

Another well to well difference may be different ligands. Another difference may be different ligand density on the particles/beads.

Thus, the invention provides a novel use of microcarrriers immobilised to solid support, namely for cell culture and/or cell screening and/or cell assaying. The cells or cell conditions may be studied with the cells attached to the microcarriers. In some cases it may be desirable to detach the cells from the microcarriers, for example for further enrichment of the cells.

The cell screening tool used in the method of the invention enables easy handling of the cells during the small scale cell growth procedure with a minimal loss of cells and therefore high yield. The screening tool also enables easy readout of the cell culture results since the tool can be placed directly under a microscope. Many results can be read in a short time. A further advantage is that the cell culture procedure may be automated.

### BRIEF DESCRIPTION OF THE DRAWINGS

FIG. 1 shows immobilised CYTODEX™ 3 microcarriers according to the invention: 6 h, 1 day and 4 days after inoculation with VERO cells.

FIG. 2 shows immobilised microcarrier CYTODEX™ 3 according to the invention: 6 days after inoculation with VERO cells.

FIG. 3 shows VERO cells cultivated in a conventional spinner flask on microcarrier CYTODEX™ 3.

### DEFINITIONS

The term “microcarrier” means a particulate material, such as a bead or sphere. The purpose of using a particulate material is to expand the available surface area to a 3 dimensional structure for the cells to grow/expand on.

The term “small cell culture” means a number of cells in the range of approximately 10² to 10⁸.

The term “cell culture media” means any media suitable for culture of a desired cell.

The term “stem cells” means any stem cell, preferably human adult or embryonal stem cells.

The term “affinity ligands” in the context of the immobilised microcarrier of the invention means any compound or coating of the microcarriers that have affinity for the desired cells, for example it could be an antibody, part of an antibody, aptamer, lectin, protein, peptide, amino acid or synthetic molecule.

### DETAILED DESCRIPTION OF THE INVENTION

The invention will described below in association with some detailed embodiments which only are given to exemplify and not limit the invention.

In the presently preferred embodiment, a screening tool is used which is produced by coating microcarriers on polystyrene support for small scale cell culture.

The invention will now be described by coating of CYTODEX™ microcarrier beads onto polystyrene support, exemplified by a Petri dish.

A sterile polystyrene Petri dish is coated with CYTODEX™ beads by contacting a slurry comprising CYTODEX™ beads in a solvent with the Petri dish. After a few minutes drying in air non-entrapped particles were washed off from the supports by flushing with a wash bottle, first with ethanol and then with water. For the production of the screening tool reference is made to co-owned WO 9411421. The only difference is that the screening tool according to the present invention uses microcarriers for cell culture, such as CYTODEX™, instead of the particles mentioned in WO 1994/011421. Alternatively, a slurry of beads in acetone/PBS is used for coating and then the beads are dried in a fume hood until the liquid has evaporated (approximately 1-2 hours). The plates are then carefully washed and dried in an oven (50-70° C.) over night.

The CYTODEX™ beads are now immobilised to the surfaces of the Petri dish. The beads may optionally be provided with cell specific ligands, such as arginine.

The mircocarriers in the Petri dishes are conserved with for example glycerol. Before inoculation the Petri dishes are washed 3 times with PBS and once with cell culture medium. Inoculum may be prepared in tissue culture flasks. The cells are detached using for example 0.02% EDTA.

### EXAMPLES

The present examples are provided for illustrative purposes only, and should not be construed as limiting the invention as defined by the appended claims. All references given below and elsewhere in the present specification are hereby included herein via reference.

### Cell Culture Experiment

An inoculum of VERO cells was provided onto a Petri dish with immobilised microcarriers prepared according to the invention. The inoculum concentration was 5.88 E5 cells/ml corresponding to about 1.25 E5 cells per cm². The cells were grown in an incubator in an atmosphere containing 7% CO₂.

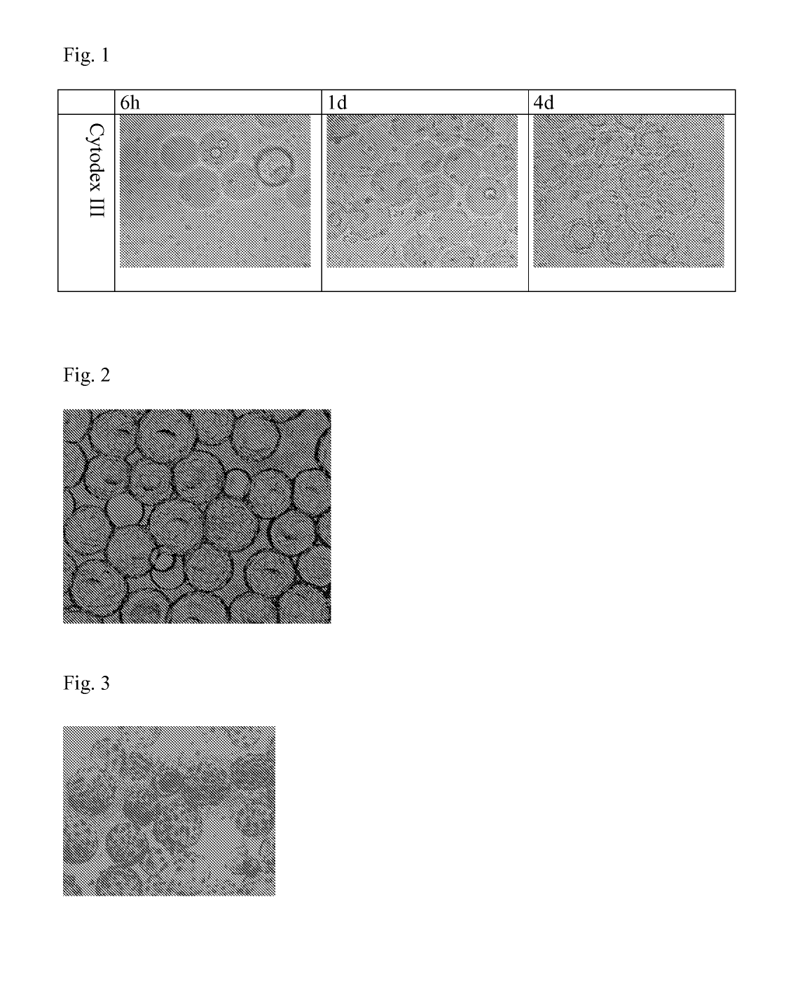

Cells grown on the microcarrier coated support according to the invention show very promising behaviour, see FIG. 1-2, compared to conventional cell culture in a spinner flask culture, see FIG. 3. The cells are similar in morphology after growth on conventional microcarriers, such as CYTODEX™, in spinner flasks and on a microcarrier coated support according to the invention. The new format of cell culture of the invention makes it very useful as a cell screening tool.

The above examples illustrate specific aspects of the present invention and are not intended to limit the scope thereof in any respect and should not be so construed. Those skilled in the art having the benefit of the teachings of the present invention as set forth above, can effect numerous modifications thereto. These modifications are to be construed as being encompassed within the scope of the present invention as set forth in the appended claims.

1: A method for small scale cell culture, comprising adding cells to particulate matter immobilised on a solid phase and growing said cells on said particulate matter.

2: The method of claim 1, wherein the particulate matter is microcarriers in the form of beads or spheres.

3: The method of claim 1, wherein the particulate matter is made of synthetic or natural polymers or inorganic materials.

4: The method of claim 3, wherein the particulate matter is cell culture beads.

5: The method of claim 1, wherein the solid phase is a molded polymer article.

6: The method of claim 1, wherein the solid phase is made of or coated with polystyrene, styrene-acrylonitrile copolymer, styrene maleic anhydride copolymer, poly vinyl chloride resin etc.

7: The method of claim 1, wherein particulate matter is attached to the solid phase by mechanical interlocking or interdiffusion of polymer chains.

8: The method of claim 7, wherein the solid phase comprises a surface or coating capable of being at least partially dissolved/swollen in a solvent.

9: The method of claim 1, wherein particulate matter is attached to the solid phase by chemical or biological bonding to the solid support.

10: The method of claim 1, wherein particulate matter are attached to the solid phase by (hydrophobic) interaction to the solid support.

11: The method of claim 1, wherein particulate matter is attached to the solid phase via a liquid adhesive to an inert surface.

12: The method of claim 1, wherein the cells are mammalian, bacterial or yeast cells.

13: The method of claim 1, wherein the cells are stem cells.

14: The method of claim 1, wherein the particulate matter is provided with ligands having any kind of interaction, such as affinity, for specific cells or cell structures.

15: The method of claim 14, wherein the ligands are selected from synthetic or natural ligands.

16: The method of claim 1, wherein the particulate matter is coated with an adhesion factor, such as gelatine, fibronectin, laminin, collagen, vitronectin or tenascin.

17: The method of claim 1, wherein the solid phase is microtiter plate with a plurality of wells each provided with immobilised particles.

18: The method of claim 1, wherein the particulate matter are beads made of dextran, cellulose or polyethylene.

19: The method of claim 17, wherein the cell culture conditions differ in one or more of the wells.

20: The method of claim 17, wherein the cell culture material differs in one or more of the wells of the microtiter plate.

21: The method of claim 20, wherein the difference is different ligands.

22: The method of claim 20, wherein the difference is a difference in ligand density on the particulate matter.

23: A cell screening tool comprising:
a solid surface made of or coated with polystyrene, styrene-acrylonitrile copolymer, styrene maleic anhydride copolymer, poly vinyl chloride resin; and
microcarriers for cell culture immobilised on said surface.

24: The cell screening tool of claim 23, wherein said microcarriers are provided with cell specific ligands.

25: The cell screening tool of claim 23, wherein the microcarriers are CYTODEX™.

26-27. (canceled)
